(19) United States
(12) Patent Application Publication
(10) Pub. No.: US 2025/0100963 A1
(43) Pub. Date: Mar. 27, 2025

(54) SULFONATED AND PHOSPHORYLATED AROMATIC MOLECULES

(71) Applicant: Xavier University of Louisiana, New Orleans, LA (US)

(72) Inventor: Rami A. AL-HORANI, New Orleans, LA (US)

(21) Appl. No.: 18/891,013

(22) Filed: Sep. 20, 2024

Related U.S. Application Data
(60) Provisional application No. 63/584,402, filed on Sep. 21, 2023.

(51) Int. Cl. C07C 309/59; A61K 31/17; A61K 31/663; C07F 9/38

(52) U.S. Cl. CPC C07C 309/59; A61K 31/17; A61K 31/663; C07F 9/3882

(57) ABSTRACT

The present disclosure relates to methods of use and treatment by inhibition of Cathepsin G by compounds described herein.

### CROSS REFERENCE TO RELATED APPLICATIONS

This patent application claims priority to U.S. Provisional Application No. 63/584,402 filed Sep. 21, 2023. The disclosure of the priority application is incorporated in its entirety herein by reference.

### FIELD OF THE INVENTION

The present disclosure relates methods of use and treatment by inhibition of Cathepsin G.

### BACKGROUND

Human cathepsin G (CatG) belongs to a family of cationic serine proteases, which was first identified in the azurophilic granules of neutrophil leukocytes. CatG has a dual trypsin- as well as chymotrypsin-like specificity with preference to Lys, Phe, Arg or Leu as P1 substrate residue. Human CatG is biosynthesized in the form of a 255-amino acid inactive precursor, which contains a signal peptide, an activation peptide at the N-terminus, and a C-terminal extension. The catalytic activity of CatG depends on a catalytic triad of Ser195, Asp102 and His57. CatG is a degradative enzyme that acts intracellularly to digest pathogens and extracellularly to breakdown extracellular matrix components at inflammation sites. CatG has also been reported to activate receptors, platelets, and angiotensin I, amongst others. There is a need in the art for inhibitors of CatG.

### BRIEF SUMMARY

An embodiment of the present disclosure can comprise a compound of Formula I:

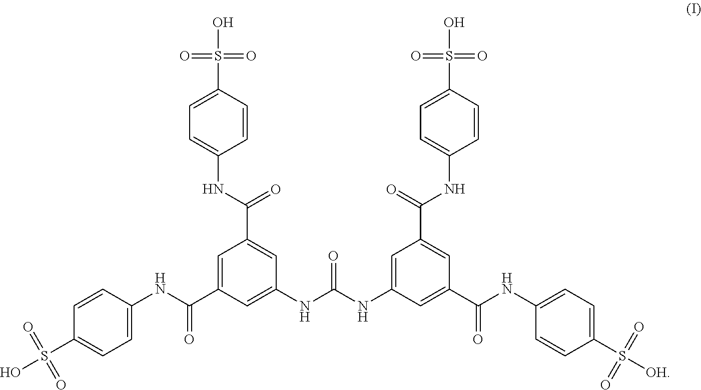 [SMILES: O=C(Nc1cc(C(=O)Nc2ccc(S(=O)(=O)O)cc2)cc(C(=O)Nc2ccc(S(=O)(=O)O)cc2)c1)Nc1cc(C(=O)Nc2ccc(S(=O)(=O)O)cc2)cc(C(=O)Nc2ccc(S(=O)(=O)O)cc2)c1]

An embodiment of the present disclosure can comprise compound of Formula II:

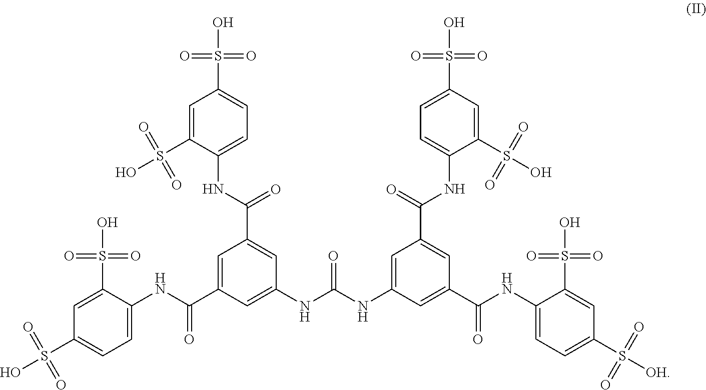 [SMILES: O=C(Nc1cc(C(=O)Nc2ccc(S(=O)(=O)O)cc2S(=O)(=O)O)cc(C(=O)Nc2ccc(S(=O)(=O)O)cc2S(=O)(=O)O)c1)Nc1cc(C(=O)Nc2ccc(S(=O)(=O)O)cc2S(=O)(=O)O)cc(C(=O)Nc2ccc(S(=O)(=O)O)cc2S(=O)(=O)O)c1]

An embodiment of the present disclosure can comprise a compound of Formula III:

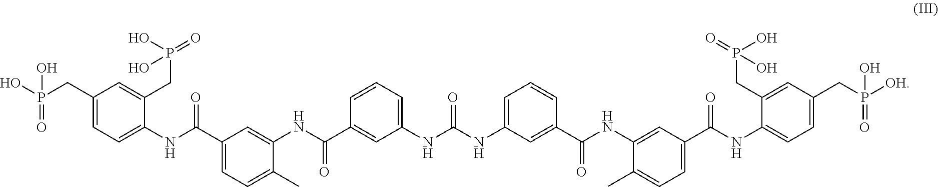 [SMILES: Cc1ccc(C(=O)Nc2ccc(CP(=O)(O)O)cc2CP(=O)(O)O)cc1NC(=O)c1cccc(NC(=O)Nc2cccc(C(=O)Nc3cc(C(=O)Nc4ccc(CP(=O)(O)O)cc4CP(=O)(O)O)ccc3C)c2)c1]

In an embodiment, the composition can comprise a composition comprising an effective amount of a compound of Formula 1.

In an embodiment, the composition can comprise a composition comprising an effective amount of a compound of Formula 2.

In an embodiment, the composition can comprise a composition comprising an effective amount of a compound of Formula 3.

In an embodiment, the composition can comprise a composition comprising an effective amount of a combination of any of the compounds of Formula 1, 2 or 3.

In an embodiment, the composition can comprise a composition comprising an effective amount of a compound of Formula 1, 2, 3, or any combination thereof, wherein the composition is a pharmaceutical composition.

In an embodiment, the composition can be formulated as a liquid, semi-solid, capsule, powder, pill, gel, or paste.

In an embodiment, the composition can be formulated as a liquid, semi-solid, capsule, powder, pill, gel, or paste, and optionally the composition is a pharmaceutical composition, that is formulated to be administered by subcutaneous, intramuscular, intravenous, intraperitoneal, vaginal, rectal, intrapleural, intravesicular, intrathecal, topical, nasal, inhalation, oral administration, or a combination of routes.

In an embodiment, the composition can be a pharmaceutical composition further comprising a pharmaceutically acceptable excipient, carrier, diluent, vehicle, adjuvant, or a combination thereof.

In an embodiment, the composition can be a composition wherein the effective amount of the compound is between about 1 μg and 1,000 μg.

In an embodiment, the composition can be a composition wherein the effective amount of the compound is between about 1 μg and 100 μg, 50 μg and 250 μg, 200 μg and 500 μg, 300 μg and 700 μg, 400 μg and 800 μg, 500 μg and 1,000 μg.

In an embodiment, the composition can be a composition wherein the effective amount of the compound is about 1, 2, 3, 4, 5, 6, 7, 8, 9, 10, 20, 30, 40, 50, 60, 70, 80, 90, 100, 110, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000 μg.

In an embodiment, the composition can be a composition, wherein the effective amount of the compound is between about 1 mg and 1,000 mg.

In an embodiment, the composition can be a composition, wherein the effective amount of the compound is between about 1 mg and 100 mg, 50 mg and 250 mg, 200 mg and 500 mg, 300 mg and 700 mg, 400 mg and 800 mg, 500 mg and 1,000 mg.

In an embodiment, the composition can be a composition, wherein the effective amount of the compound is about 1, 2, 3, 4, 5, 6, 7, 8, 9, 10, 20, 30, 40, 50, 60, 70, 80, 90, 100, 110, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000 mg.

In an embodiment, the composition can be a composition, wherein the effective amount of the compound is about between about 1 g and 1,000 g.

In an embodiment, the composition can be a composition, wherein the effective amount of the compound is 1, 2, 3, 4, 5, 6, 7, 8, 9, 10, 20, 30, 40, 50, 60, 70, 80, 90, 100, 110, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000 g.

An embodiment can comprise a method for treating inflammation in a subject comprising administering an effective amount of a compound described herein.

In an embodiment, a method for the treatment of an inflammatory disease in a subject can comprise administering an effective amount of a compound described herein.

In an embodiment, a method for treating an inflammatory disease in a subject can comprise administering an effective amount of a compound or a composition described herein, wherein the inflammatory disease is periodontitis, psoriasis, rheumatoid arthritis, ischemic reperfusion injury, coronary artery disease, bone metastasis, acute respiratory distress syndrome, chronic obstructive pulmonary disease, cystic fibrosis, or any combination thereof.

In an embodiment, a method for the treatment of inflammation in a subject or an inflammatory disease in a subject, wherein the composition is formulated for subcutaneous, intramuscular, intravenous, intraperitoneal, vaginal, rectal, intrapleural, intravesicular, intrathecal, topical, nasal, inhalation, oral administration, or a combination of routes.

In an embodiment, a method for the treatment of inflammation in a subject or an inflammatory disease in a subject, wherein the compound inhibits protease.

In an embodiment, a method for the treatment of inflammation in a subject or an inflammatory disease in a subject, wherein the compound inhibits CatG-mediated degradation of laminin.

In an embodiment, a method for the treatment of inflammation in a subject or an inflammatory disease in a subject, wherein the compound inhibits CatG-mediated degradation of fibronectin.

### BRIEF DESCRIPTION OF THE DRAWINGS

FIG. 1 depicts the chemical types of claimed inhibitors of human CatG.

FIG. 2 depicts the chemical structures of inhibitors of Formulae I, II, and III.

FIG. 3 depicts the inhibition profiles of the inhibitors of Formulae I, II, and III.

FIG. 4 depicts a graph of Salt-dependent direct inhibition of CatG by inhibitor of Formula II, using the corresponding chromogenic substrate (S-7388) hydrolysis assay. Salt concentrations used are 100 mM (●) and 200 mM (◯).

FIG. 5 depicts a graph of Michaelis-Menten kinetics of the substrate hydrolysis by human CatG in the presence of different concentrations of inhibitor of Formula II.

FIG. 6 depicts the effect of inhibitor of Formula II on CatG-mediated hydrolysis of laminin as evaluated by SDS-PAGE under reducing conditions. Inhibitor of Formula II concentrations used were 0, 10, 100 and 1000 μM.

FIG. 7 depicts the effect of inhibitor of Formula II on CatG-mediated hydrolysis of fibronectin as evaluated by SDS-PAGE under reducing conditions. Inhibitor of Formula II concentrations used were 0, 10, 100 and 1000 μM.

### DETAILED DESCRIPTION

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

### Definitions

Unless defined otherwise, all technical and scientific terms used herein have the same meaning as commonly understood to one of ordinary skill in the art to which the presently disclosed subject matter belongs.

Following long-standing patent law convention, the terms “a,” “an,” and “the” refer to “one or more” when used in this application, including the claims.

The term “and/or” when used in describing two or more items or conditions, refers to situations where all named items or conditions are present or applicable, or to situations wherein only one (or less than all) of the items or conditions is present or applicable.

“Cathepsin G,” as used herein, refers broadly to a protein that in humans is encoded by the CTSG gene.

“Inflammatory disease,” as used herein, refers broadly to when the subject's immune system attacks the body's own tissues. Examples of inflammatory disease include but are not limited to periodontitis, psoriasis, rheumatoid arthritis, ischemic reperfusion injury, coronary artery disease, bone metastasis, acute respiratory distress syndrome, chronic obstructive pulmonary disease, cystic fibrosis.

“Non-human mammals,” as used herein, refers broadly to non-human mammalian animals, including but not limited to dogs, cats, mice, rats, guinea pigs, rabbits, ferrets, cows, horses, sheep, goats, and pigs. Non-human mammals can be mammalian pets or companion animals, including but not limited to dogs and cats and also mice, rats, guinea pigs, ferrets, and rabbits. The non-human mammal can be a dog or a cat.

“Treatment” refers broadly to both therapeutic treatment and prophylactic or preventative measures. Those in need of treatment include those already with the disorder as well as those in which the disorder is to be prevented. As used herein, the term “treating,” refers broadly to treating a disease, arresting, or reducing the development of the disease or its clinical symptoms, and/or relieving the disease, causing regression of the disease or its clinical symptoms. Therapy encompasses prophylaxis, treatment, remedy, reduction, alleviation, and/or providing relief from a disease, signs, and/or symptoms of a disease. Therapy encompasses an alleviation of signs and/or symptoms in patients with ongoing disease signs and/or symptoms. Therapy also encompasses “prophylaxis”. The term “reduced”, for purpose of therapy, refers broadly to the clinical significant reduction in signs and/or symptoms. Therapy includes treating relapses or recurrent signs and/or symptoms. Therapy encompasses but is not limited to precluding the appearance of signs and/or symptoms anytime as well as reducing existing signs and/or symptoms and eliminating existing signs and/or symptoms. Therapy includes treating chronic disease (“maintenance”) and acute disease. For example, treatment includes treating or preventing relapses or the recurrence of signs and/or symptoms.

“Effective amount,” as used herein, refers broadly to the amount of an agent, e.g., a compound, antibody, vector or cells that, when administered to a patient for treating a disease, is sufficient to affect such treatment for the disease. The effective amount can be an amount effective for prophylaxis, and/or an amount effective for prevention. The effective amount can be an amount effective to reduce, an amount effective to prevent the incidence of signs/symptoms, to reduce the severity of the incidence of signs/symptoms, to eliminate the incidence of signs/symptoms, to slow the development of the incidence of signs/symptoms, to prevent the development of the incidence of signs/symptoms, and/or effect prophylaxis of the incidence of signs/symptoms. The “effective amount” can vary depending on the disease and its severity and the age, weight, medical history, susceptibility, and pre-existing conditions, of the patient to be treated. The term “effective amount” is synonymous with “therapeutically effective amount” for purposes described herein.

“Mammal,” as used herein, refers broadly to any and all warm-blooded vertebrate animals of the class Mammalia, characterized by a covering of hair on the skin and, in the female, milk-producing mammary glands for nourishing the young. Mammals include, but are not limited to, humans, domestic and farm animals, and zoo, sports, or pet animals. Similarly, the term “subject” or “patient” includes both human and veterinary subjects and/or patients.

### Human Cathepsin G (CatG) Inhibitors

The physiological activity of Human cathepsin G (CatG) is regulated by α2-macroglobulin, serpin B1, α1-antichymotrypsin, α1-protienase inhibitor, proteinase inhibitor 6, and secretory leukocyte protease inhibitor. Given the wide substrate specificity of CatG, it contributes to many diseases such as periodontitis, rheumatoid arthritis, ischemic reperfusion injury, coronary artery disease and bone metastasis. It is also implicated in acute respiratory distress syndrome, chronic obstructive pulmonary disease, cystic fibrosis and even pain.

Despite the promise of CatG inhibition in treating and/or managing many diseases, few inhibitors have been developed including small molecules, peptides, aptamers and sulphated saccharides. In particular, small molecule CatG inhibitors include organophosphorus derivatives, boswellic acid derivatives, 2-substituted saccharines, thiadiazolidinone dioxides and N-arylacyl O-sulfonated aminoglycosides, whilst sulfated saccharides include heparin and its derivatives.

Mechanistically, organophosphorus derivatives, boswellic acid derivatives, 2-substituted saccharines and thiadiazolidinone dioxides appear to be active site inhibitors, however, N-arylacyl O-sulfonated aminoglycosides were reported to be partial mixed inhibitors of CatG with IC50 values of 0.42-209 μM. Importantly, heparin, an anticoagulant sulfated glycosaminoglycan, was characterized as allosteric inhibitor with an estimated Ki of <25 pM. Although the potency of heparin inhibiting CatG is remarkable, its anti-CatG mediated anti-inflammatory activity is of limited clinical utility given the high risk of excessive bleeding.

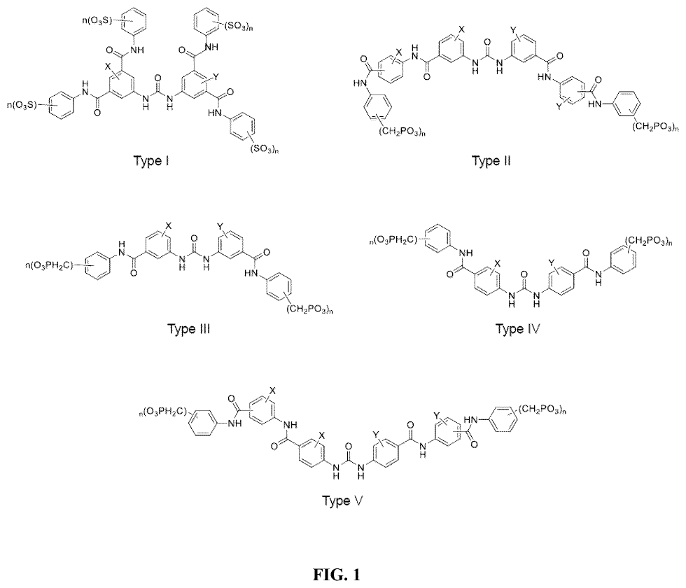

Sulfonated aromatic molecules with the general structures (Types I-V) in FIG. 1 may serve as potent and allosteric inhibitors of CatG with no significant risk of bleeding, given their unique bleeding free-anticoagulant mechanism of factor XIa (FXIa) inhibition. Sulfonated compounds inhibit CatG in a salt-dependent fashion with IC50 values in nanomolar range and efficacies of ˜90% under physiological conditions. They also demonstrated significant selectivity over other digestive and other clotting serine proteases. Their activity was also found to be physiologically relevant as they did protect fibronectin and laminin against CatG-mediated degradation. Similar to heparin and its derivatives, Michaelis-Menten kinetics indicated that they are allosteric inhibitors of CatG. The described CatG inhibitors are synthesized in high yields. The inhibitors are tested using chromogenic substrate hydrolysis assay to determine their IC50's. Their physiological relevance was established by a host of other assays. Overall, the described inhibitors offer a great promise for developing novel small molecules as therapeutics for treatment of periodontitis, psoriasis, rheumatoid arthritis, ischemic reperfusion injury, coronary artery disease and bone metastasis. They can also be implicated in acute respiratory distress syndrome, chronic obstructive pulmonary disease, cystic fibrosis and even pain. There is a need for allosteric inhibitors of CatG with no significant risk of bleeding.

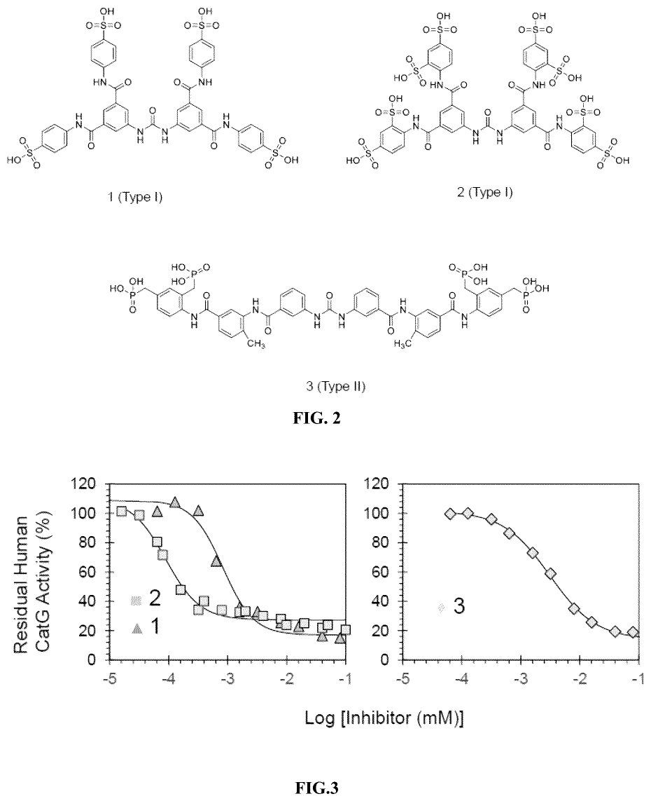

The compounds described herein comprise one of the above types with the following structural features to act as CatG inhibitors for the treatment of the above-mentioned conditions. Compounds of Formulae I, II, and III are depicted in FIG. 2 along with their corresponding types.

A method of treating inflammation in a subject in need thereof can comprise administering at an effective amount of at least one inhibitor of Cathepsin G (CatG) comprising a compound of Formulae I, II, III, or a combination thereof. The subject can be a mammal. The subject can be a human.

### Cathepsin GA Allosteric Inhibitor Compounds

The CatG inhibitor compound can comprise an allosteric inhibitor of CatG. The CatG inhibitor compound can comprise a compound of Formula I:

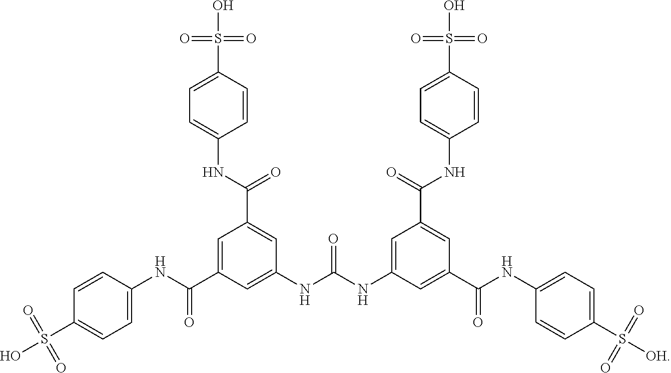 [SMILES: O=C(Nc1cc(C(=O)Nc2ccc(S(=O)(=O)O)cc2)cc(C(=O)Nc2ccc(S(=O)(=O)O)cc2)c1)Nc1cc(C(=O)Nc2ccc(S(=O)(=O)O)cc2)cc(C(=O)Nc2ccc(S(=O)(=O)O)cc2)c1]

The CatG inhibitor compound can comprise a compound of Formula II:

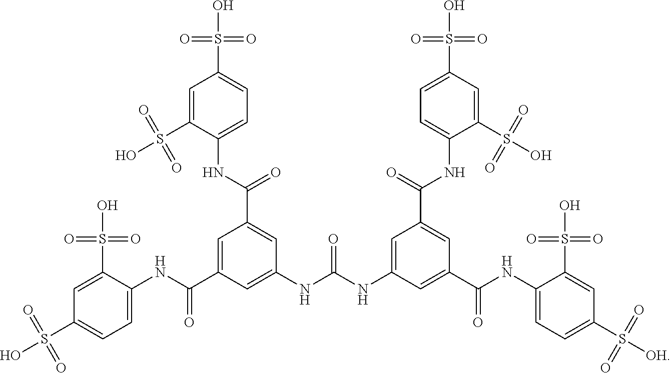 [SMILES: O=C(Nc1cc(C(=O)Nc2ccc(S(=O)(=O)O)cc2S(=O)(=O)O)cc(C(=O)Nc2ccc(S(=O)(=O)O)cc2S(=O)(=O)O)c1)Nc1cc(C(=O)Nc2ccc(S(=O)(=O)O)cc2S(=O)(=O)O)cc(C(=O)Nc2ccc(S(=O)(=O)O)cc2S(=O)(=O)O)c1]

The CatG inhibitor compound can comprise a compound of Formula III:

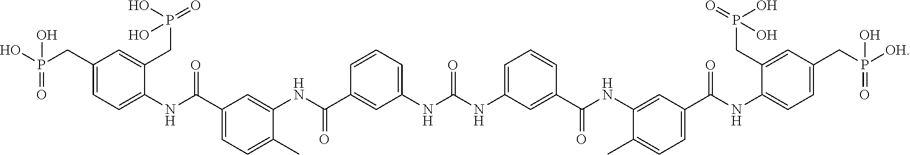 [SMILES: Cc1ccc(C(=O)Nc2ccc(CP(=O)(O)O)cc2CP(=O)(O)O)cc1NC(=O)c1cccc(NC(=O)Nc2cccc(C(=O)Nc3cc(C(=O)Nc4ccc(CP(=O)(O)O)cc4CP(=O)(O)O)ccc3C)c2)c1]

The CatG inhibitor compound described herein can be a compound of Formula I, II, or III.

### Methods of Use

The CatG inhibitor compound of Formula I, II, or III can be used in methods and compositions for the treatment of inflammation, optionally an inflammatory disease. An inflammatory disease can comprise any disease wherein a subject's immune response attacks the subject's tissue. The inflammatory disease can be periodontitis, psoriasis, rheumatoid arthritis, ischemic reperfusion injury, coronary artery disease, bone metastasis, acute respiratory distress syndrome, chronic obstructive pulmonary disease, cystic fibrosis and any combination thereof.

A method for the treatment of inflammation can comprise administering an effective amount of a compound of Formula I, II, III, or a combination thereof.

A method for the treatment of an inflammatory disease can comprise administering an effective amount of a compound of Formula I, II, III, or a combination thereof. The inflammatory disease can be periodontitis, psoriasis, rheumatoid arthritis, ischemic reperfusion injury, coronary artery disease, bone metastasis, acute respiratory distress syndrome, chronic obstructive pulmonary disease, cystic fibrosis and any combination thereof.

Compositions for the treatment of inflammation can comprise an effective amount of a compound of Formula I, II, III, or a combination thereof.

Compositions for the treatment of an inflammatory disease can comprise an effective amount of a compound of Formula I, II, III, or a combination thereof. The inflammatory disease can be periodontitis, psoriasis, rheumatoid arthritis, ischemic reperfusion injury, coronary artery disease, bone metastasis, acute respiratory distress syndrome, chronic obstructive pulmonary disease, cystic fibrosis and any combination thereof.

The compound of Formula I, II, III, or a combination thereof can be formulated into a composition, optionally, a pharmaceutical composition. Compositions comprising an effective amount of a compound of Formula I, II, III, or a combination thereof can be used in methods for treating inflammation. Compositions comprising an effective amount of a compound of Formula I, II, III, or a combination thereof can be used in methods for treating an inflammatory disease. The inflammatory disease can be periodontitis, psoriasis, rheumatoid arthritis, ischemic reperfusion injury, coronary artery disease, bone metastasis, acute respiratory distress syndrome, chronic obstructive pulmonary disease, cystic fibrosis and any combination thereof.

The compound of Formula I, II, III, or a combination thereof, can be used in the manufacture of a medicament for the treatment of inflammation.

The compound of Formula I, II, III, or a combination thereof, can be used in the manufacture of a medicament for the treatment of an inflammatory disease. The inflammatory disease can be periodontitis, psoriasis, rheumatoid arthritis, ischemic reperfusion injury, coronary artery disease, bone metastasis, acute respiratory distress syndrome, chronic obstructive pulmonary disease, cystic fibrosis and any combination thereof.

It was discovered that the compounds of Formula I, II, III, or a combination thereof, can be used in the treatment of inflammation without serious side effects. The anionic characteristic of the compounds confers low cellular, fetal, and central nervous system toxicity arising from their highly charged nature which prevents passive diffusion through cell membrane, placenta, and blood brain barrier. Furthermore, 10 UM of these compounds did not affect the cell viability of three cell lines (MCF-7, HEK-293, and CaCo-2).

The method further comprises inhibition of proteases. Direct inhibition of CatG was determined at pH 7.4 and 37° C. by a chromogenic substrate hydrolysis assay, as described previously [15]. To each well of a 96-well microplate containing 88 μl of 20 mM tris buffer containing 100 mM NaCl, 2.5 mM CaCl2, 0.1% PEG 8000 and 0.05% tween 80 was added 3 μl of CatG (final concentration of 30 nM), and 5 μl of H2O or potential inhibitor (final concentration of 0-100 μM). Following a 5-min incubation, 3 μl of CatG substrate was added (final concentration 750 μM) and the residual CatG activity was obtained from the initial rate of increase of absorbance at 405 nm. The relative residual activity of CatG at each of the inhibitor concentrations was obtained from the ratio of CatG activity in the presence and absence of the inhibitor. Logistic Equation (1) was used to plot the dose-dependence curve to obtain the IC50 (potency) and efficacy of CatG inhibition. Here, Y is the ratio of residual CatG activity in the presence of inhibitor to that in its absence, Y_{O} and Y_{M} are the minimum and maximum values of fractional residual CatG activity, respectively, IC50 is the concentration of the inhibitor that results in 50% inhibition of CatG activity and HS is the Hill slope.

The method further comprises inhibition of CatG-mediated degradation of laminin. Inhibition of CatG cleavage of laminin by sulfonated or phosphorylated compounds was studied using SDSPAGE. Briefly, CatG (0.8 μM) was incubated with different concentrations of the inhibitor (final concentrations; 0 μM, 10 μM, 100 M and 1000 μM), and laminin (20 μg). Following incubation for 60 min, the samples were quenched using SDS-PAGE loading buffer containing DTT and subjected to electrophoresis on 10% SDS-PAGE pre-cast gels. The gels were visualized by silver staining. Although the sulfonated or phosphorylated compounds inhibit CatG hydrolysis of chromogenic substrate, extracellular matrix (ECM) components serve as more relevant substrates of CatG. In fact, based on their in vitro properties, it was reasoned that these inhibitors could protect extracellular matrix components from proteolysis mediated by proteinases activated during the inflammatory process including CatG. Accordingly, the in vitro effect of compound of Formula II on the CatG-mediated proteolysis of laminin, a non-collagenous component of ECM and basement membranes was studied. SDS-PAGE analyses showed that laminin was significantly cleaved by CatG, as evidenced by the disappearance of the ≥200-kDa laminin bands. In the presence of inhibitor 2 (10-1000 μM), however, laminin is protected completely from cleavage by CatG. Thus, SDS-PAGE analyses indicate that sulfonated molecule inhibition of CatG is physiologically relevant.

The method further comprises inhibition of CatG-mediated degradation of fibronectin. Inhibition of CatG cleavage of fibronectin by sulfonated or phosphorylated molecules was studied using SDS-PAGE. Briefly, CatG (0.5 μM) was incubated with different concentrations of the inhibitor (final concentrations; 0 μM, 10 μM, 100 μM, and 1000 μM), and fibronectin (36 nM). Following incubation for 60 mins, the samples were quenched using SDS-PAGE loading buffer containing DTT and subjected to electrophoresis on 10% SDSPAGE pre-cast gels. The gels were visualized by silver staining. Although sulfonated or phosphorylated compounds inhibit CatG hydrolysis of chromogenic substrate, extracellular matrix (ECM) components serve as more relevant substrates of CatG. In fact, based on their in vitro properties, it was reasoned that these inhibitors could protect extracellular matrix components from proteolysis mediated by proteinases activated during the inflammatory process including CatG. Accordingly, the in vitro effect of the inhibitor on the CatG-mediated proteolysis of fibronectin, a non-collagenous component of ECM and basement membranes was studied. SDS-PAGE analyses showed that fibronectin was significantly cleaved by CatG, as evidenced by the disappearance of the ≥200-kDa fibronectin bands. In the presence of the inhibitor (10-1000 μM), however, fibronectin is protected completely from cleavage by CatG. Thus, SDS-PAGE analyses indicate that sulfonated compounds inhibition of CatG is physiologically relevant.

### Pharmaceutical Compositions

The disclosure further provides pharmaceutical compositions comprising a compound described herein. Pharmaceutical compositions described herein can comprise a single compound described herein or a combination of the compounds described herein. Pharmaceutical compositions described herein typically comprise at least one pharmaceutically acceptable excipient (e.g., one or more than one pharmaceutically acceptable excipient).

The pharmaceutically acceptable excipient includes, but is not limited to, a binder, filler, diluent, disintegrant, wetting agent, lubricant, glidant, coloring agent, dyemigration inhibitor, sweetening agent, flavoring agent or a combination thereof.

Binders or granulators impart cohesiveness to a tablet to ensure the tablet remaining intact after compression. Binders or granulators include, but are not limited to, starches, such as corn starch, potato starch, and pre-gelatinized starch (e.g., STARCH 1500®); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, PA); and mixtures thereof.

Fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder is hydroxypropylcellulose.

Diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets. The diluent is lactose monohydrate. The diluent is lactose monohydrate Fast-Flo 316 NF.

Disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the compositions described herein can vary. The disintegrant is croscarmellose sodium. The disintegrant is croscarmellose sodium NF (Ac-Di-Sol).

Lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, MD) and CAB-O-SIL® (Cabot Co. of Boston, MA); and mixtures thereof. The lubricant is magnesium stearate.

Glidants include colloidal silicon dioxide, CAB-O-SIL® (Cabot Co. of Boston, MA), and talc, including asbestos-free talc.

Coloring agents include any of the approved, certified, water soluble FD&C dyes, and water insoluble FD&C dyes suspended on alumina hydrate, and color lakes and mixtures thereof.

Flavoring agents include natural flavors extracted from plants, such as fruits, and synthetic blends of compounds that provide a pleasant taste sensation, such as peppermint and methyl salicylate.

Sweetening agents include sucrose, lactose, mannitol, syrups, glycerin, sucralose, and artificial sweeteners, such as saccharin, stevioside (Stevia) and aspartame.

Emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether.

Solvents include glycerin, sorbitol, ethyl alcohol, and syrup.

Examples of non-aqueous liquids utilized in emulsions include mineral oil and cottonseed oil. Organic acids include citric and tartaric acid. Sources of carbon dioxide include sodium bicarbonate and sodium carbonate.

Pharmaceutical compositions can be formulated for administration by a variety of means including orally, parenterally, by inhalation spray, topically, or rectally in formulations containing pharmaceutically acceptable carriers, adjuvants and vehicles. The term “parenteral” as used here includes subcutaneous, intravenous, intramuscular, and intraarterial injections with a variety of infusion techniques. Intraarterial and intravenous injection as used herein includes administration through catheters.

Pharmaceutical compositions can be formulated in accordance with the routine procedures adapted for desired administration route. Accordingly, the compositions described herein can take such forms as suspensions, solutions or emulsions in oily or aqueous vehicles, and can contain formulatory agents such as suspending, stabilizing and/or dispersing agents. The compositions described herein can be formulated as a preparation for implantation or injection. Thus, for example, the compositions described herein can be formulated with polymeric or hydrophobic materials (e.g., as an emulsion in an acceptable oil) or ion exchange resins, or as sparingly soluble derivatives (e.g., as a sparingly soluble salt). The compounds described herein and the compositions described herein can be in powder form for constitution with a vehicle, e.g., sterile pyrogen-free water, before use. Formulations for each of these methods of administration can be found, for example, in Remington: The Science and Practice of Pharmacy, A. Gennaro, ed., 20th edition, Lippincott, Williams & Wilkins, Philadelphia, PA.

The compositions described herein can be formulated for oral administration. These compositions can comprise solid, semisolid, gelmatrix or liquid dosage forms. As used herein, oral administration includes buccal, lingual, and sublingual administration. Oral dosage forms include, without limitation, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, syrups or any combination thereof. The compositions described herein can be formulated for oral administration are in the form of a tablet or a capsule. The composition described herein is in a form of a tablet. The composition described herein is in a form of a capsule. The compound described herein is contained in a capsule.

The capsules are immediate release capsules. A non-limiting example of a capsule is a coni-snap® hard gelatin capsule.

The compositions described herein can be in the form of compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which can be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. A film coating can impart the same general characteristics as a sugar coating. Multiple compressed tablets are compressed tablets made by more than one compression cycle, including layered tablets, and press-coated or dry coated tablets.

The coating is a film coating. The film coating comprises Opadry White and simethicone emulsion 30% USP.

The compounds described herein can be contained in a tablet. The compounds described herein can be contained in a compressed tablet. The compounds described herein can be contained in a film-coated compressed tablet. A pharmaceutical composition described herein can be in the form of film-coated compressed tablet.

Pharmaceutical compositions described herein can be in the form of soft or hard capsules, which can be made, for example, from gelatin, methylcellulose, starch, or calcium alginate. The hard gelatin capsule, also known as the dry-filled capsule (DFC), can comprise of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells can contain a preservative to prevent the growth of microorganisms. Preservatives are those as described herein, including methyl- and propyl-parabens, and sorbic acid. The liquid, semisolid, and solid dosage forms provided herein can be encapsulated in a capsule. Liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules can also be coated as known by those of skill in the art in order to modify or sustain dissolution of the active ingredient.

The pharmaceutical compositions described herein can be in liquid or semisolid dosage forms, including emulsions, solutions, suspensions, elixirs, and syrups. An emulsion can be a two-phase system, in which one liquid is dispersed in the form of small globules throughout another liquid, which can be oil-in-water or water-in-oil. Emulsions can include a pharmaceutically acceptable non-aqueous liquids or solvent, emulsifying agent, and preservative. Suspensions can include a pharmaceutically acceptable suspending agent and preservative. Aqueous alcoholic solutions can include a pharmaceutically acceptable acetal, such as a di-(lower alkyl) acetal of a lower alkyl aldehyde, e.g., acetaldehyde diethyl acetal; and a water-miscible solvent having one or more hydroxyl groups, such as propylene glycol and ethanol. Elixirs can be clear, sweetened, and hydroalcoholic solutions. Syrups can be concentrated aqueous solutions of a sugar, for example, sucrose, and can comprise a preservative. For a liquid dosage form, for example, a solution in a polyethylene glycol can be diluted with a sufficient quantity of a pharmaceutically acceptable liquid carrier, e.g., water, to be measured conveniently for administration.

The pharmaceutical compositions described herein for oral administration can be also provided in the forms of liposomes, micelles, microspheres, or nanosystems. Micellar dosage forms can be prepared as described in U.S. Pat. No. 6,350,458.

The pharmaceutical compositions described herein can be provided as non-effervescent or effervescent, granules and powders, to be reconstituted into a liquid dosage form. Pharmaceutically acceptable carriers and excipients used in the non-effervescent granules or powders can include diluents, sweeteners, and wetting agents. Pharmaceutically acceptable carriers and excipients used in the effervescent granules or powders can include organic acids and a source of carbon dioxide.

Coloring and flavoring agents can be used in all of the above dosage forms. Flavoring and sweetening agents are especially useful in the formation of chewable tablets and lozenges.

The pharmaceutical compositions described herein can be formulated as immediate or modified release dosage forms, including delayed-, extended, pulsed-, controlled, targeted-, and programmed-release forms.

The pharmaceutical compositions described herein comprise a film coating.

The pharmaceutical compositions described herein can comprise another active ingredient that does not impair the composition's therapeutic or prophylactic efficacy or can comprise a substance that augments or supplements the composition's efficacy.

The pharmaceutical compositions described herein can be in a modified release or a controlled release dosage form. The compositions described herein can comprise particles exhibiting a particular release profile. For example, the composition described herein can comprise a compound described herein in an immediate release form while also comprising a statin or a pharmaceutically acceptable salt, solvate, ester, amide, or prodrug thereof in a modified release form, both compressed into a single tablet. Other combination and modification of release profile can be achieved as understood by one skilled in the art. Examples of modified release dosage forms suited for pharmaceutical compositions of the instant disclosure are described, without limitation, in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

The compositions described herein are a matrix-controlled release dosage form. The release profile of the compound described herein and of the other pharmaceutically active agent is the same or different. Matrix-controlled release dosage forms are described, for example, in Takada et al in “Encyclopedia of Controlled Drug Delivery,” Vol. 2, Mathiowitz ed., Wiley, 1999.

The erodible matrix of the matrix-controlled release form comprises chitin, chitosan, dextran, or pullulan; gum agar, gum arabic, gum karaya, locust bean gum, gum tragacanth, carrageenans, gum ghatti, guar gum, xanthan gum, or scleroglucan; starches, such as dextrin or maltodextrin; hydrophilic colloids, such as pectin; phosphatides, such as lecithin; alginates; propylene glycol alginate; gelatin; collagen; cellulosics, such as ethyl cellulose (EC), methylethyl cellulose (MEC), carboxymethyl cellulose (CMC), carrrboxymethyl ethyl cellulose (CMEC) hydroxyethyl cellulose (HEC), hydroxypropyl cellulose (HPC), cellulose acetate (CA), cellulose propionate (CP), cellulose butyrate (CB), cellulose acetate butyrate (CAB), cellulose acetate phthalate (CAP), cellulose acetate trimellitate (CAT), hydroxypropyl methyl cellulose (HPMC), HPMCP, HPMCAS, hydroxypropyl methyl cellulose acetate trimellitate (HPMCAT), or ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, NJ); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; or other acrylic acid derivatives, such as homopolymers and copolymers of butylmethacrylate, methylmethacrylate, ethylmethacrylate, ethylacrylate, (2-dimethylaminoethyl) methacrylate, or (trimethylaminoethyl) methacrylate chloride; or any combination thereof.

The pharmaceutical compositions described herein are in a matrix-controlled modified release form comprising a non-erodible matrix. The statin, the compound described herein is dissolved or dispersed in an inert matrix and is released primarily by diffusion through the inert matrix once administered. The non-erodible matrix of the matrix-controlled release form comprises an insoluble polymer, such as polyethylene, polypropylene, polyisoprene, polyisobutylene, polybutadiene, polymethylmethacrylate, polybutylmethacrylate, chlorinated polyethylene, polyvinylchloride, a methyl acrylate-methyl methacrylate copolymer, an ethylene-vinylacetate copolymer, an ethylene/propylene copolymer, an ethylene/ethyl acrylate copolymer, a vinylchloride copolymer with vinyl acetate, a vinylidene chloride, an ethylene or a propylene, an ionomer polyethylene terephthalate, a butyl rubber epichlorohydrin rubber, an ethylene/vinyl alcohol copolymer, an ethylene/vinyl acetate/vinyl alcohol terpolymer, an ethylene/vinyloxyethanol copolymer, a polyvinyl chloride, a plasticized nylon, a plasticized polyethyleneterephthalate, a natural rubber, a silicone rubber, a polydimethylsiloxane, a silicone carbonate copolymer, or a hydrophilic polymer, such as an ethyl cellulose, a cellulose acetate, a crospovidone, or a cross-linked partially hydrolyzed polyvinyl acetate; a fatty compound, such as a carnauba wax, a microcrystalline wax, or a triglyceride; or any combination thereof.

The compositions described herein that are in a modified release dosage form can be prepared by methods known to those skilled in the art, including direct compression, dry or wet granulation followed by compression, melt-granulation followed by compression.

The administration can be by oral, parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarenal, intraretinal, intraspinal, intrasynovial, intrathoracic, intrauterine, intravesical, bolus, vaginal, rectal, buccal, sublingual, intranasal, iontophoretic means, or transdermal means.

### Dosages

The effective amount of the CatG inhibitor compounds described herein can be between about 1 μg and 1,000 μg.

The effective amount of the CatG inhibitor compounds described herein can be between about 1 μg and 100 μg, 50 μg and 250 μg, 200 μg and 500 μg, 300 μg and 700 μg, 400 μg and 800 μg, 500 μg and 1,000 μg.

The effective amount of the CatG inhibitor compounds described herein can be about 1, 2, 3, 4, 5, 6, 7, 8, 9, 10, 20, 30, 40, 50, 60, 70, 80, 90, 100, 110, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000 μg.

The effective amount of the CatG inhibitor compounds described herein can be between about 1 mg and 1,000 mg.

The effective amount of the CatG inhibitor compounds described herein can be between about 1 mg and 100 mg, 50 mg and 250 mg, 200 mg and 500 mg, 300 mg and 700 mg, 400 mg and 800 mg, 500 mg and 1,000 mg.

The effective amount of the CatG inhibitor compounds described herein can be about 1, 2, 3, 4, 5, 6, 7, 8, 9, 10, 20, 30, 40, 50, 60, 70, 80, 90, 100, 110, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000 mg.

The effective amount of the CatG inhibitor compounds described herein can be about between about 1 g and 1,000 g.

The effective amount of the CatG inhibitor compounds described herein can be 1, 2, 3, 4, 5, 6, 7, 8, 9, 10, 20, 30, 40, 50, 60, 70, 80, 90, 100, 110, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000 g.

The daily dosage of the formulations described herein can be 0.001, 0.01, 0.1, 1, 2, 3, 4, 5, 6, 7, 8, 9, 10, 20, 30, 40, 50, 60, 70, 80, 90, 100, 110, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000 mg per patient, per day, or equivalent doses as determined by a practitioner to achieve a serum concentration that is clinically relevant.

### EXAMPLES

### Example 1

### Direct Inhibition of Human CatG

Direct inhibition of CatG was determined at pH 7.4 and 37° C. by a chromogenic substrate hydrolysis assay, as described previously [15]. To each well of a 96-well microplate containing 88 μl of 20 mM tris buffer containing 100 mM NaCl, 2.5 mM CaCl2, 0.1% PEG 8000 and 0.05% tween 80 was added 3 μl of CatG (final concentration of 30 nM), and 5 μl of H2O or potential inhibitor (final concentration of 0-100 μM). Following a 5-min incubation, 3 μl of CatG substrate was added (final concentration 750 μM) and the residual CatG activity was obtained from the initial rate of increase of absorbance at 405 nm. The relative residual activity of CatG at each of the inhibitor concentrations was obtained from the ratio of CatG activity in the presence and absence of the inhibitor. Logistic Equation (1) was used to plot the dose-dependence curve to obtain the IC50 (potency) and efficacy of CatG inhibition. Here, Y is the ratio of residual CatG activity in the presence of inhibitor to that in its absence, YO and YM are the minimum and maximum values of fractional residual CatG activity, respectively, IC50 is the concentration of the inhibitor that results in 50% inhibition of CatG activity and HS is the Hill slope.

$$Y = {Y}_{O} + \frac{{Y}_{M} − {Y}_{O}}{1 + {10}^{( log {( I )}_{o} − log {IC}_{50} ) ( H 5 )}} \qquad eq . 1$$

Molecules 1-3 inhibited human CatG with IC50 values in the low micromolar and submicromolar concentrations as provided in Table 1 and FIG. 3. Other inhibition parameters are also provided in the table.

TABLE 1
Inhibition parameters of human CatG by
compounds of Formula I, II, and III.^{a}

| Formula | IC₅₀(μM) | HS | ΔY (%) | (−) Charges |
|---|---|---|---|---|
| I | .083 ± 0.14^{b} | 1.7 ± 0.4 | 91.9 ± 6.8 | 4 |
| II | 0.09 ± 0.01 | 1.5 ± 0.3 | 82.5 ± 7.4 | 8 |
| III | 3.1 ± 0.19 | 1.1 ± 0.1 | 87.5 ± 1.8 | 4 |

^{a}The values of IC₅₀, HS, and ΔY were obtained following the nonlinear regression analysis of direct inhibition of human CatG in appropriate Tris buffer of pH 7.4 at 37° C. Inhibition was monitored by spectrophotometric measurement of residual enzyme activity.
^{b}Errors represent ±1 SE.

### Example 2

### Direct Inhibition of Other Proteases

Direct inhibitions of thrombin (FIIa), factor VIIa (FVIIa), factor IXa (FIXa), factor Xa (FXa), FXIa, factor XIIa (FXIIa), trypsin and chymotrypsin were evaluated using the corresponding chromogenic substrate hydrolysis assays as previously reported [15-18]. Briefly, to each well of a 96-well microplate containing 85-185 μl of pH 7.4° C. Tris buffer containing 100 mM NaCl, 2.5 mM CaCl2, 0.1% PEG 8000, and 0.05% tween 80 at 25 (thrombin) or 37° C. (FVIIa, FIXa, FXa, FXIa, FXIIa, chymotrypsin, and trypsin) were added 5 μl of enzyme (thrombin, FVIIa, FIXa, FXa, FXIa, FXIIa, chymotrypsin, and trypsin) and 5 μl of the inhibitor. Following an incubation period of 5 min, the appropriate chromogenic substrates (Spectrozyme TH, Spectrozyme FIXa, Spectrozyme FXa, S-2366, Spectrozyme FXIIa and S-2222) were added and the residual enzyme activity was measured from the initial rate of increase in absorbance at 405 nm. Relative residual enzyme activity as a function of the inhibitor concentration was fitted using Logistic Equation (1) to obtain the IC50 (potency), A Y % (efficacy) of enzyme inhibition, and HS (Hill slope). The concentrations of enzymes and substrates in microplate cells were: 6 nM and 50 μM for thrombin; 1.09 nM and 125 μM for FXa; 5 nM and 125 μM for FXIIa; 89 nM and 850 μM for FIXa; 8 nM and 1000 UM for FVIIa (along with 40 nM recombinant tissue factor); 72.5 ng/ml and 80 μM for bovine trypsin; and 500 ng/ml and 240 μM for bovine chymotrypsin.

Inhibition profiles of molecules 1-3 against several enzymes (thrombin, factor Xa, factor IXa, factor XIa, factor XIIIa, human neutrophil elastase, proteinase 3, plasmin, trypsin, and chymotrypsin) are listed below. Inhibitors demonstrated variable levels of selectivity, with the compound of Formula II appearing to be the most selective. See Tables 2 and 3.

TABLE 2
Human FXIa inhibition by sulfonated
compounds of Formula I, II, and III.^{a}

| Inhibitors | IC₅₀(μM) | HS | ΔY (%) |
|---|---|---|---|
| 1 | 29.5 ± 4.6^{b} | 1.2 ± 0.2 | 90.9 ± 5.8 |
| 2 | >500^{c} | NA^{d} | NA |
| 3 | 7.4 ± 0.9 | 1.9 ± 0.5 | 68.0 ± 3.7 |

^{a}The values of IC₅₀, HS, and ΔY were obtained via the nonlinear regression analysis of FXIa direct inhibition Tris-HCl buffer. Inhibition was followed by the spectrophotometric measurement of residual FXIa activity.
^{b}Errors represent ±1 SE.
^{c}Estimated measurement based on the highest concentration used in the study.
^{d}Not available.

TABLE 3
The inhibition of thrombin and FXa
by sulfonated molecules (1-3).^{a}′

| | Thrombin | | | FXa | | |
|---|---|---|---|---|---|---|
| Formula | IC₅₀(μM) | HS | ΔY (%) | IC₅₀(μM) | HS | ΔY (%) |
| I | >100^{b} | NA^{c} | NA | >100 | NA | NA |
| III | >500 | NA | NA | >100 | NA | NA |

^{a}IC₅₀, HS, and ΔY were obtained via the non-linear regression analysis of human FIIa and FXa in appropriate Tris-HCl buffer. Inhibition was followed by the spectrophotometric measurement of residual FIIa or FXa activity.
^{b}Estimated value based on the highest concentration used in the study.
^{c}Not available.

### Example 3

### Salt-Dependent Direct Inhibition of Human CatG

The direct inhibition of CatG cleavage of a chromogenic substrate was determined at 37° C., as described above, in pH 7.4 Tris buffer containing 2.5 mM CaCl2, 0.1% PEG 8000, 0.05% tween 80 and 50-500 mM NaCl. The Ki (μM) values were estimated using an IC50 to Ki conversion equation [19].

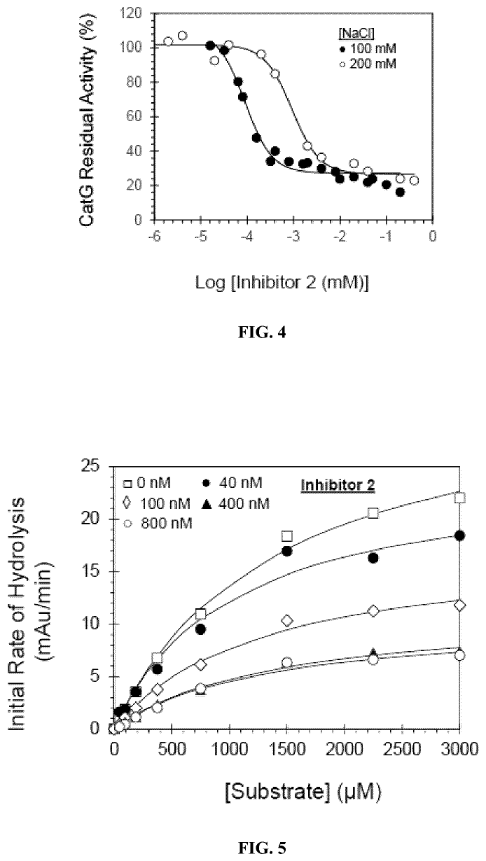

Although the sulfonated and/or phosphorylated compounds-CatG interaction is likely to be electrostatically driven, non-ionic forces may contribute to a significant extent. A significant non-ionic binding energy component increases the interaction specificity because the majority of non-ionic forces, for example, cation—π interactions, H-bonding and others depend strongly on the orientation and the distance of interacting pair of molecules [20, 21]. In contrast, ionic bonds are non-directional and less dependent on distance, which may increase initial interaction but offer less selectivity of recognition. To determine the nature of interactions between inhibitor 2 and CatG, the IC50 values were measured as a function of the ionic strength of the medium at pH 7.4 and 37° C. The IC50 values were measured spectrophotometrically under various salt concentrations (100 and 200 mM), as described above. Interestingly, a 2-fold increase in salt concentration led to a ˜10-fold decrease in the potency of inhibitor 2. Subsequently, the corresponding Ki (nM) values were estimated using a formula previously reported following a classical model of noncompetitive enzyme inhibition and the results are reported below. Overall, results indicate that anionic interactions are very important for the inhibition of CatG by the compound of Formula II. Table 4 and FIG. 4.

TABLE 4
Salt-dependent inhibition of human CatG
by the compound of Formula II.^{a}

| | | | | | Calculated |
|---|---|---|---|---|---|
| Formula | IC₅₀(μM) | HS | ΔY (%) | [NaCl] | Ki (μM) |
| II | 0.09 ± 0.01 | 1.5 ± 0.3 | 82.5 ± 7.4 | 100 mM | ~0.075 |
| | 0.92 ± 0.15 | 1.5 ± 0.3 | 75.2 ± 3.4 | 200 mM | ~0.91 |

^{a}The values of IC₅₀, HS, and ΔY were obtained via the nonlinear regression analysis of direct inhibition of human CatG in appropriate Tris-HCl buffer of pH 7.4 at 37° C. Inhibition was followed by the spectrophotometric measurement of residual enzyme activity.
^{b}Errors represent ±1 SE.

### Example 4

Michaelis-Menten kinetics. Mechanism of Inhibition

The initial rate of the hydrolysis of the chromogenic substrate by CatG was monitored from the linear increase in absorbance corresponding to less than 10% consumption of substrate at 37° C. in pH 7.4 20 mM Tris buffer containing 100 mM NaCl, 2.5 mM CaCl2, 0.1% PEG 8000 and 0.05% tween 80. The initial rate was measured at various substrate concentrations (0-2500 mM) at fixed enzyme concentration (30 nM), and fixed sulfonated compounds concentrations (0, 25, 50, 100, 200, 400, and 800 nM). The data were fitted by the Michaelis-Menten Equation (3) to determine the KM (substrate affinity) and VMAX (maximum reaction velocity).

$$V = \frac{{V}_{MAX} [ S ]}{{K}_{M} + [ S ]} \qquad eq . 3$$

To understand the mechanistic basis of inhibition, Michaelis-Menten kinetics of S-7388 hydrolysis by CatG was performed in the presence of inhibitor 2 at pH 7.4 and 37° C. FIG. 5 shows the initial rate profiles in the presence of inhibitor 2 (0-800 nM). Each curve shows a characteristic rectangular hyperbolic dependence, which could be fitted using Michaelis-Menten equation (Equation (3)) to obtain the apparent KM and VMAX (Table 5). The KM for S-7388 remained essentially unchanged in the presence or absence of inhibitor 2, whilst the VMAX decreased steadily from 33.5±1.8 mAU/min in the absence of inhibitor to 10.6±0.95 mAU/min at 800 nM of inhibitor 2 (˜3-fold). Therefore, sulfonated aromatic inhibitors appear to bring about structural changes in the active site of CatG, which does not affect the formation of Michaelis complex, but induces a significant dysfunction in the catalytic apparatus. This indicates that they are allosteric inhibitors of CatG. Table 5 and FIG. 5.

TABLE 5
Michaelis-Menten Kinetics of chromogenic substrate hydrolysis
by CatG in the presence of the compound of Formula II.^{a}

| Formula II | Kₘ | Vₘₐₓ |
|---|---|---|
| (nM) | (mM) | (mAU/min) |
| 0 | 1.44 ± 0.17^{b} | 33.5 ± 1.8 |
| 40 | 1.20 ± 0.3 | 26.1 ± 2.5 |
| 100 | 1.29 ± 0.19 | 17.6 ± 1.1 |
| 400 | 1.40 ± 0.22 | 11.4 ± 0.8 |
| 800 | 1.32 ± 0.207 | 10.6 ± 0.95 |

^{a}K_{M} and V_{MAX} values of the chromogenic substrate hydrolysis by CatG were measured as described herein. mAU indicates milliabsorbance units.
^{b}Errors represent ±1 SE.

### Example 5

### Effect of Inhibitor on CatG-Mediated Degradation of Laminin

Inhibition of CatG cleavage of laminin by sulfonated compounds was studied using SDSPAGE. Briefly, CatG (0.8 μM) was incubated with different concentrations of the inhibitor (final concentrations; 0 μM, 10 μM, 100 μM and 1000 μM), and laminin (20 μg). Following incubation for 60 min, the samples were quenched using SDS-PAGE loading buffer containing DTT and subjected to electrophoresis on 10% SDS-PAGE pre-cast gels. The gels were visualized by silver staining.

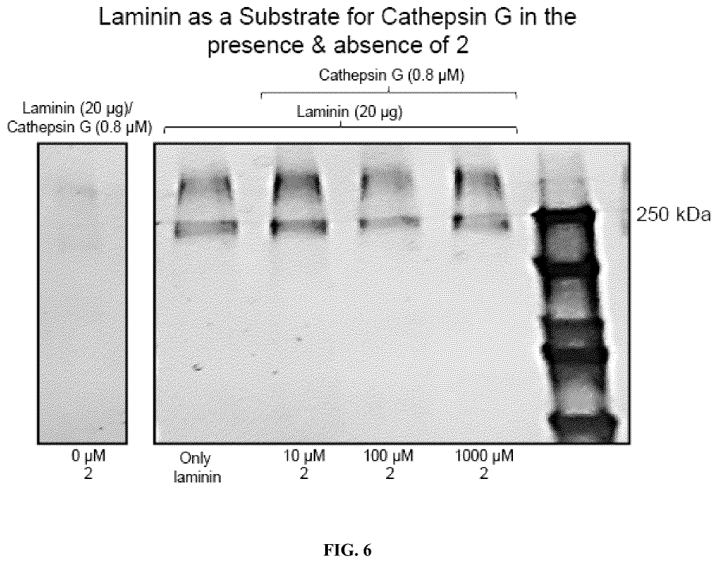

Although the compounds of Formula I, II, and III inhibit CatG hydrolysis of chromogenic substrate, extracellular matrix (ECM) components serve as more relevant substrates of CatG. In fact, based on their in vitro properties, it was reasoned that these inhibitors could protect extracellular matrix components from proteolysis mediated by proteinases activated during the inflammatory process including CatG. Accordingly, the in vitro effect of compound of Formula II on the CatG-mediated proteolysis of laminin, a non-collagenous component of ECM and basement membranes was studied. SDS-PAGE analyses showed that laminin was significantly cleaved by CatG, as evidenced by the disappearance of the ≥200-kDa laminin bands (FIG. 6, only laminin and 0 μM lanes). In the presence of inhibitor 2 (10-1000 μM), however, laminin is protected completely from cleavage by CatG. Thus, SDS-PAGE analyses indicate that sulfonated molecule inhibition of CatG is physiologically relevant. See FIG. 6.

### Example 6

### Effect of Inhibitor on CatG-Mediated Degradation of Fibronectin

Inhibition of CatG cleavage of fibronectin by sulfonated and phosphorylated molecules was studied using SDS-PAGE. Briefly, CatG (0.5 μM) was incubated with different concentrations of the inhibitor (final concentrations; 0 μM, 10 μM, 100 μM, and 1000 μM), and fibronectin (36 nM). Following incubation for 60 mins, the samples were quenched using SDS-PAGE loading buffer containing DTT and subjected to electrophoresis on 10% SDSPAGE pre-cast gels. The gels were visualized by silver staining.

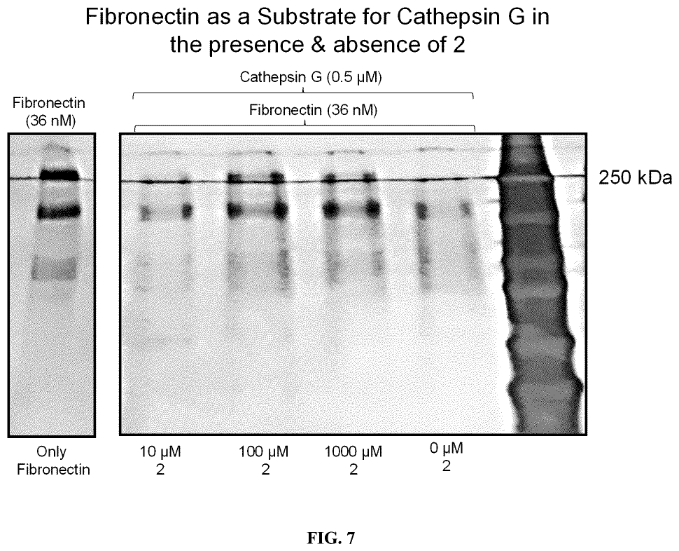

Although sulfonated compounds inhibit CatG hydrolysis of chromogenic substrate, extracellular matrix (ECM) components serve as more relevant substrates of CatG. In fact, based on their in vitro properties, it was reasoned that these inhibitors could protect extracellular matrix components from proteolysis mediated by proteinases activated during the inflammatory process including CatG. Accordingly, the in vitro effect of the inhibitor on the CatG-mediated proteolysis of fibronectin, a non-collagenous component of ECM and basement membranes was studied. SDS-PAGE analyses showed that fibronectin was significantly cleaved by CatG, as evidenced by the disappearance of the ≥200-kDa fibronectin bands (FIG. 7, only fibronectin and 0 μM lanes). In the presence of the inhibitor (10-1000 μM), however, fibronectin is protected completely from cleavage by CatG. Thus, SDS-PAGE analyses indicate that sulfonated compounds inhibition of CatG is physiologically relevant. See FIG. 7.

TABLE 6
GatC Inhibitor Compounds

| | CATG |
|---|---|
| | IC₅₀ |
| Inhibitor | (μM) |
| 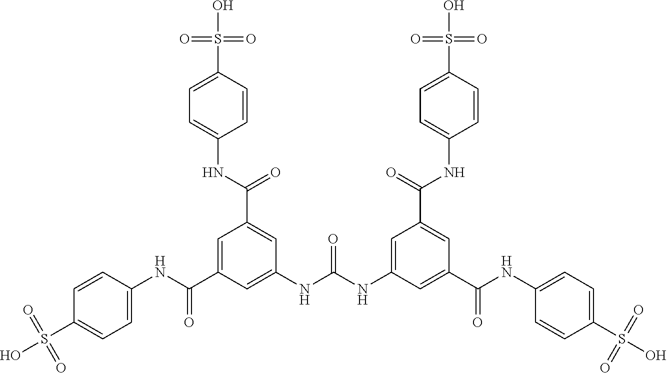 [SMILES: O=C(Nc1cc(C(=O)Nc2ccc(S(=O)(=O)O)cc2)cc(C(=O)Nc2ccc(S(=O)(=O)O)cc2)c1)Nc1cc(C(=O)Nc2ccc(S(=O)(=O)O)cc2)cc(C(=O)Nc2ccc(S(=O)(=O)O)cc2)c1] | 0.83 |
| 4,4′,4″,4″-((5,5′-(carbonylbis(azanediyl))bis(isophthaloyl))tetrakis(azanediyl))tetrabenzenesulfonic acid | |
| (Formula I) | |
| 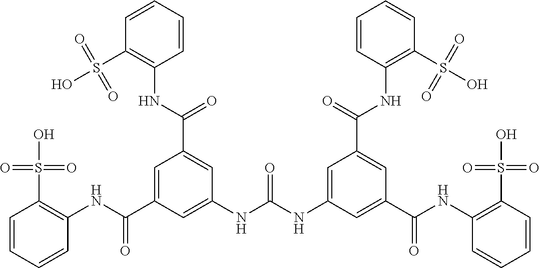 [SMILES: O=C(Nc1cc(C(=O)Nc2ccccc2S(=O)(=O)O)cc(C(=O)Nc2ccccc2S(=O)(=O)O)c1)Nc1cc(C(=O)Nc2ccccc2S(=O)(=O)O)cc(C(=O)Nc2ccccc2S(=O)(=O)O)c1] | 1-5 |
| 2,2′,2″,2″′-((5,5′- | |
| (carbonylbis(azanediyl))bis(isophthaloyl))tetrakis(azanediyl))tetrabenzenesulfonic acid | |
| 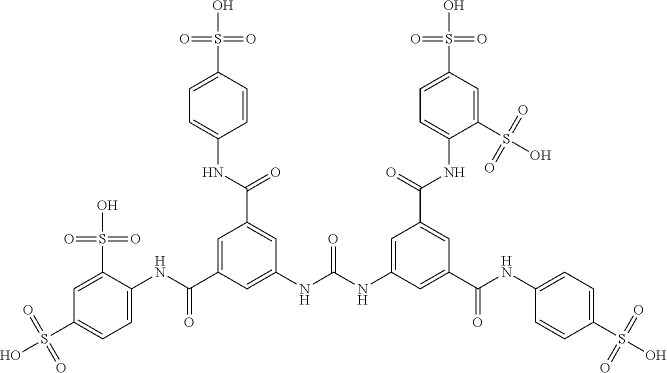 [SMILES: O=C(Nc1cc(C(=O)Nc2ccc(S(=O)(=O)O)cc2)cc(C(=O)Nc2ccc(S(=O)(=O)O)cc2S(=O)(=O)O)c1)Nc1cc(C(=O)Nc2ccc(S(=O)(=O)O)cc2)cc(C(=O)Nc2ccc(S(=O)(=O)O)cc2S(=O)(=O)O)c1] | 0.5-1 |
| 4,4′-((3,3′-(carbonylbis(azanediyl))bis(5-((4- | |
| sulfophenyl)carbamoyl)benzoyl))bis(azanediyl))bis(benzene-1,3-disulfonic acid) | |
| 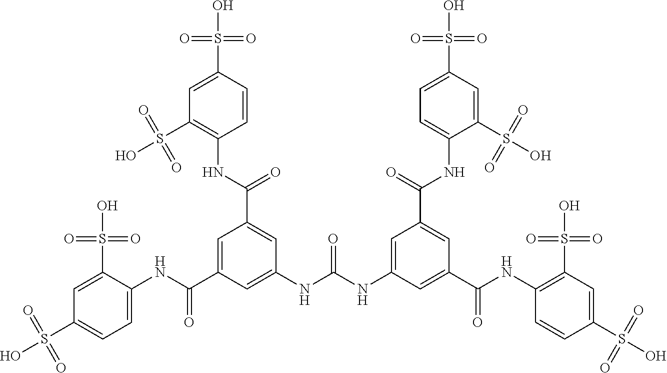 [SMILES: O=C(Nc1cc(C(=O)Nc2ccc(S(=O)(=O)O)cc2S(=O)(=O)O)cc(C(=O)Nc2ccc(S(=O)(=O)O)cc2S(=O)(=O)O)c1)Nc1cc(C(=O)Nc2ccc(S(=O)(=O)O)cc2S(=O)(=O)O)cc(C(=O)Nc2ccc(S(=O)(=O)O)cc2S(=O)(=O)O)c1] | 0.09 |
| 4,4′,4″,4′-((5,5′- | |
| (carbonylbis(azanediyl))bis(isophthaloyl))tetrakis(azanediyl))tetrakis(benzene-1,3- | |
| disulfonic acid) (Formula II) | |
| 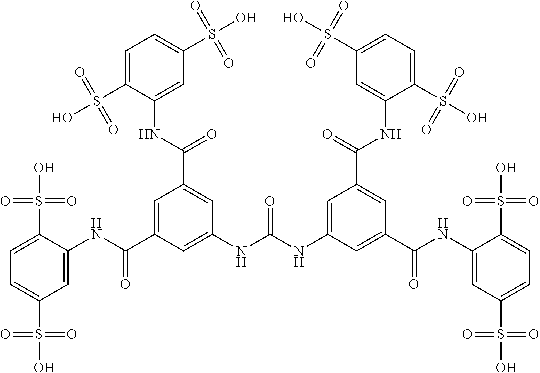 [SMILES: O=C(Nc1cc(C(=O)Nc2cc(S(=O)(=O)O)ccc2S(=O)(=O)O)cc(C(=O)Nc2cc(S(=O)(=O)O)ccc2S(=O)(=O)O)c1)Nc1cc(C(=O)Nc2cc(S(=O)(=O)O)ccc2S(=O)(=O)O)cc(C(=O)Nc2cc(S(=O)(=O)O)ccc2S(=O)(=O)O)c1] | 1-5 |
| 2,2′,2″,2″′-((5,5′- | |
| (carbonylbis(azanediyl))bis(isophthaloyl))tetrakis(azanediyl))tetrakis(benzene-1,4- | |
| disulfonic acid) | |
| 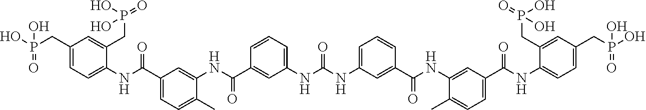 [SMILES: Cc1ccc(C(=O)Nc2ccc(CP(=O)(O)O)cc2CP(=O)(O)O)cc1NC(=O)c1cccc(NC(=O)Nc2cccc(C(=O)Nc3cc(C(=O)Nc4ccc(CP(=O)(O)O)cc4CP(=O)(O)O)ccc3C)c2)c1] | 3.1 |
| ((((3,3′-((3,3′-(carbonylbis(azanediyl))bis(benzoyl))bis(azanediyl))bis(4- | |
| methylbenzoyl))bis(azanediyl))bis(benzene-4,1,3- | |
| triyl))tetrakis(methylene))tetrakis(phosphonic acid) (Formula III) | |
| 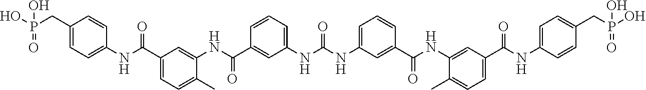 [SMILES: Cc1ccc(C(=O)Nc2ccc(CP(=O)(O)O)cc2)cc1NC(=O)c1cccc(NC(=O)Nc2cccc(C(=O)Nc3cc(C(=O)Nc4ccc(CP(=O)(O)O)cc4)ccc3C)c2)c1] | 10-100 |
| ((((3,3′-((3,3′-(carbonylbis(azanediyl))bis(benzoyl))bis(azanediyl))bis(4- | |
| methylbenzoyl))bis(azanediyl))bis(4,1-phenylene))bis(methylene))bis(phosphonic acid) | |
| 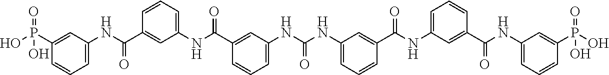 [SMILES: O=C(Nc1cccc(C(=O)Nc2cccc(C(=O)Nc3cccc(P(=O)(O)O)c3)c2)c1)Nc1cccc(C(=O)Nc2cccc(C(=O)Nc3cccc(P(=O)(O)O)c3)c2)c1] | 1-10 |
| (((3,3′-((3,3′- | |
| (carbonylbis(azanediyl))bis(benzoyl))bis(azanediyl))bis(benzoyl))bis(azanediyl))bis(3,1- | |
| phenylene))bis(phosphonic acid) | |
| 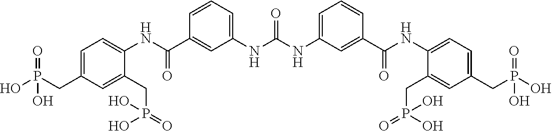 [SMILES: O=C(Nc1cccc(C(=O)Nc2ccc(CP(=O)(O)O)cc2CP(=O)(O)O)c1)Nc1cccc(C(=O)Nc2ccc(CP(=O)(O)O)cc2CP(=O)(O)O)c1] | 1-10 |
| ((((3,3′-(carbonylbis(azanediyl))bis(4-methylbenzoyl))bis(azanediyl))bis(benzene-4,1,3- | |
| triyl))tetrakis(methylene))tetrakis(phosphonic acid) | |
| 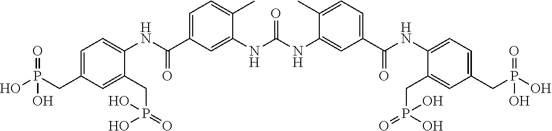 [SMILES: Cc1ccc(C(=O)Nc2ccc(CP(=O)(O)O)cc2CP(=O)(O)O)cc1NC(=O)Nc1cc(C(=O)Nc2ccc(CP(=O)(O)O)cc2CP(=O)(O)O)ccc1C] | 10-100 |
| ((((3,3′-(carbonylbis(azanediyl))bis(4-methylbenzoyl))bis(azanediyl))bis(4,1- | |
| phenylene))bis(methylene))bis(phosphonic acid) | |

What is claimed:

1. A compound of Formula I: 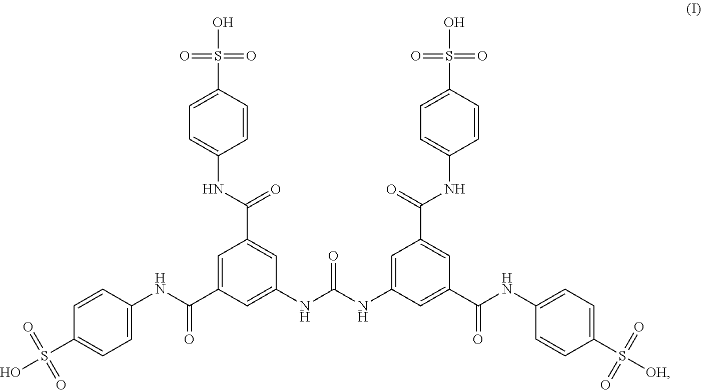 [SMILES: O=C(Nc1cc(C(=O)Nc2ccc(S(=O)(=O)O)cc2)cc(C(=O)Nc2ccc(S(=O)(=O)O)cc2)c1)Nc1cc(C(=O)Nc2ccc(S(=O)(=O)O)cc2)cc(C(=O)Nc2ccc(S(=O)(=O)O)cc2)c1]
Formula II: 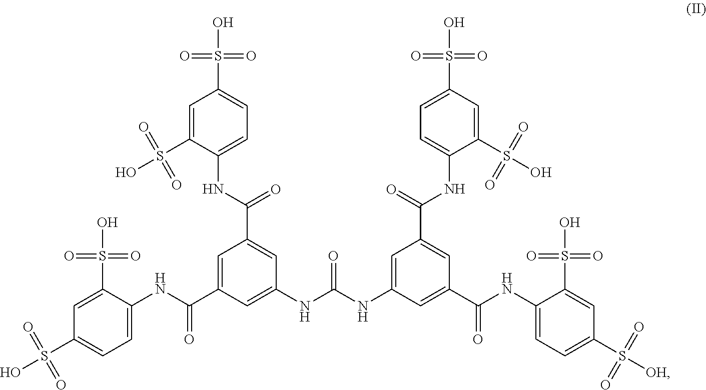 [SMILES: O=C(Nc1cc(C(=O)Nc2ccc(S(=O)(=O)O)cc2S(=O)(=O)O)cc(C(=O)Nc2ccc(S(=O)(=O)O)cc2S(=O)(=O)O)c1)Nc1cc(C(=O)Nc2ccc(S(=O)(=O)O)cc2S(=O)(=O)O)cc(C(=O)Nc2ccc(S(=O)(=O)O)cc2S(=O)(=O)O)c1]
or Formula III:

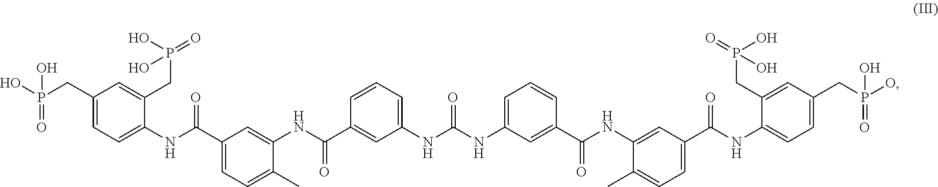

2. A composition comprising an effective amount of the compound of claim 1.

3. A composition comprising an effective amount of a combination of the compounds of claim 1.

4. The composition of claim 1, wherein the composition is a pharmaceutical composition.

5. The composition of claim 1, wherein the composition is formulated as a liquid, semi-solid, capsule, powder, pill, gel, or paste.

6. The composition of claim 1, wherein the composition is formulated to be administered by subcutaneous, intramuscular, intravenous, intraperitoneal, vaginal, rectal, intrapleural, intravesicular, intrathecal, topical, nasal, inhalation, oral administration, or a combination of routes.

7. The composition of claim 1, wherein the composition is a pharmaceutical composition further comprising a pharmaceutically acceptable excipient, carrier, diluent, vehicle, adjuvant, or a combination thereof.

8. The composition of claim 1, wherein the effective amount of the compound is between about 1 μg and 1,000 μg.

9. The composition of claim 1, wherein the effective amount of the compound is between about 1 mg and 1,000 g.

10. A method for treating inflammation in a subject comprising administering an effective amount the compound of claim 1.

11. A method for the treatment of an inflammatory disease in a subject comprising administering an effective amount the compound of any one of claim 1.

12. The method of claim 11, wherein the inflammatory disease is periodontitis, psoriasis, rheumatoid arthritis, ischemic reperfusion injury, coronary artery disease, bone metastasis, acute respiratory distress syndrome, chronic obstructive pulmonary disease, cystic fibrosis, or any combination thereof.

13. The method of claim 10, wherein the composition is formulated for subcutaneous, intramuscular, intravenous, intraperitoneal, vaginal, rectal, intrapleural, intravesicular, intrathecal, topical, nasal, inhalation, oral administration, or a combination of routes.

14. The method of claim 10, wherein the compound inhibits protease.

15. The method of claim 10, wherein the compound inhibits CatG-mediated degradation of laminin.

16. The method of claim 10, wherein the compound inhibits CatG-mediated degradation of fibronectin.

17. The method of claim 11, wherein the composition is formulated for subcutaneous, intramuscular, intravenous, intraperitoneal, vaginal, rectal, intrapleural, intravesicular, intrathecal, topical, nasal, inhalation, oral administration, or a combination of routes.

18. The method of claim 11, wherein the compound inhibits protease.

19. The method of claim 11, wherein the compound inhibits CatG-mediated degradation of laminin.

20. The method of claim 11, wherein the compound inhibits CatG-mediated degradation of fibronectin.
